(19) United States
(12) Patent Application Publication
(10) Pub. No.: US 2013/0284181 A1
(43) Pub. Date: Oct. 31, 2013

(54) ENDOTRACHEAL TUBE SYSTEM

(71) Applicant: Phillip Benjamin Guerra, Arlington, TX (US)

(72) Inventor: Phillip Benjamin Guerra, Arlington, TX (US)

(21) Appl. No.: 13/855,420

(22) Filed: Apr. 2, 2013

Related U.S. Application Data
(63) Continuation-in-part of application No. 13/550,666, filed on Jul. 17, 2012, now Pat. No. 8,485,194.
Application No. 13/550,666 is a continuation of application No. 13/168,948, filed on Jun. 25, 2011, now Pat. No. 8,220,461.
(60) Provisional application No. 61/438,465, filed on Feb. 1, 2011.
(60) Provisional application No. 61/621,782, filed on Apr. 9, 2012.

(51) Int. Cl. A61M 16/04

(52) U.S. Cl. CPC A61M 16/0463
USPC 128/207.14

(57) ABSTRACT

An endotracheal tube system includes an endotracheal tube having a first conduit and a second conduit, the first conduit being fluidly separable from the second conduit and a connector in communication with both the first conduit and the second conduit.

### BACKGROUND

1. Field of the Invention

This present application relates generally to medical devices, and more specifically, to an endotracheal (ET) tube system with means for delivering local anesthesia.

2. Description of Related Art

Tracheal intubation, typically referred to as intubation, is the placement of a flexible elastomeric tube into the trachea (windpipe) to maintain an open airway and/or to serve as a conduit for administering anesthesia and/or other substances. Intubation is generally performed on critically injured or anesthetized patients so as to reduce the possibility of asphyxiation or airway obstruction.

The most widely used route is the orotracheal passage, in which the ET tube passes through the mouth to the trachea, whereas, in a nasotracheal procedure, the ET tube passes through the nose to the trachea. Intubation is normally facilitated by using a conventional laryngoscope, flexible fiberoptic bronchoscope or video laryngoscope to identify the glottis, although other devices and techniques are available.

After the trachea has been intubated, a balloon cuff located on a distal end of the ET tube is inflated. Balloon cuffs have proven to be effective means for securing the ET tube in relative fixed position within the trachea, resulting in little to no leakage of respiratory gases and protection of the tracheobronchial tree from harmful substances such as stomach acid. Thereafter, the ET tube is secured to the face or neck and connected to a T-piece, anesthesia breathing circuit, bag valve mask device, a mechanical ventilator, and/or other operably associated device.

U.S. Pat. No. 4,669,463 to McConnell is one known embodiment indicative of a current state-of-the-art ET tube. FIGS. 1A and 1B depict exemplary oblique views of one or more features of the McConnell device. In FIG. 1A, the ET tube is shown with the capability of administering local anesthesia, while FIG. 1B shows a balloon cuff associated with the ET tube. The ET tube 11 comprises of a primary passage 13 in communication with a breathing circuit (not shown) and/or other operably associated devices at a distal end 15. The breathing circuit secures to ET tube 11 via a connector 17. Conventional connectors 17 are effective and commonly used means for securing tube 11 to a breathing circuit. ET tube 11 is further provided with a secondary passage 19, referred to as a lumen, extending from primary passage 13. An adapter 21 is in communication with the second passage 19, which in turn is in channels gas/liquid to the Murphy's eye 23. An optional balloon cuff 25 can be utilized to secure ET tube 11 in a relative fixed position while disposed within the trachea.

Although ET tube 11 provides effective means for opening the windpipe and for administering local anesthesia, considerable shortcomings remain. For example, problems arise when utilizing a secondary passage, as shown in the McConnell ET tube configuration, that remains exposed during the operation. The secondary tube is susceptible to being damaged during the operation, resulting in potentially catastrophic danger to the patient. Furthermore, the McConnell design has an increased manufacturing cost associated with extending the secondary tube outside the primary tube.

### DESCRIPTION OF THE DRAWINGS

The novel features believed characteristic of the invention are set forth in the appended claims. However, the invention itself, as well as a preferred mode of use, and further objectives and advantages thereof, will best be understood by reference to the following detailed description when read in conjunction with the accompanying drawings, wherein:

FIGS. 1A and 1B are oblique views of known ET tubes;

FIG. 2 is a perspective view of one embodiment of the invention;

FIG. 3 is a side elevational view of the device illustrated in FIG. 2;

FIG. 4 is a bottom plan view of the device illustrated in FIG. 2;

FIG. 5 is a front elevational view of the device illustrated in FIG. 2;

FIG. 6 is a side elevational view of the device shown in FIG. 2, illustrated as used with an anesthesia breathing connector;

FIG. 7 is a side elevational view of the device shown in FIG. 2, illustrated as used with a suction tube;

FIG. 8A is a perspective view of an alternate embodiment of the invention, illustrated as used with a nasal cannula;

FIG. 8B is a partial perspective view of the device shown in FIG. 2, illustrated as used with a nasal cannula;

FIG. 8C is a partial perspective view of an alternate embodiment of the invention, illustrated as used with a nasal cannula;

FIG. 9 is an exploded perspective view of an alternate embodiment of the invention;

FIG. 10 is an exploded perspective view of another alternate embodiment of the invention;

FIG. 11 is a perspective view of the embodiment illustrated in FIG. 8A;

FIG. 12 is a perspective view of one half of the tubular element of the device illustrated in FIG. 11;

FIG. 13 is a front elevational view of the device illustrated in FIG. 11;

FIG. 14 is an exploded perspective view of the device illustrated in FIG. 11;

FIG. 15 is a perspective view of yet another embodiment of the invention having a suction conduit and a breathing conduit;

FIG. 16 is a perspective view of an alternate embodiment of the invention;

FIG. 17 is a front perspective view of the device illustrated in FIG. 16;

FIG. 18 is a partial view of one half of the tubular element of the device illustrated in FIG. 16;

FIG. 19 is a front elevational view of the device illustrated in FIG. 16;

FIG. 20 is an exploded perspective view of the device illustrated in FIG. 16;

FIG. 21 is an exploded front perspective view of yet another embodiment of the invention, illustrating multi-lumen extruded tubing;

FIG. 22 is an exploded back perspective view of the device illustrated in FIG. 21;

FIG. 23 is an exploded perspective view of yet another embodiment of the invention,

FIG. 24 is an alternate exploded perspective view of the embodiment of FIG. 23;

FIG. 25 is a perspective view of the embodiment of FIG. 23 as assembled;

FIGS. 26-33 are various views of an ET tube system according to one preferred embodiment of the present application; and

FIGS. 31-33 are cross-sectional views of different embodiments of a connector of the ET tube system according to FIG. 26.

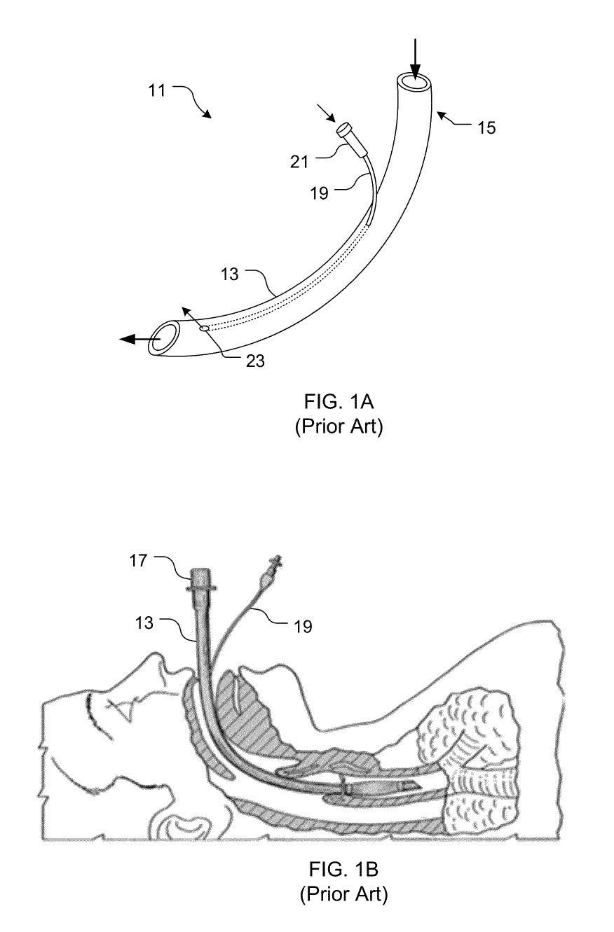

While the systems and methods according to the present application are susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the ET tube system, oral airway, and methods of use are provided below. It will of course be appreciated that in the development of any actual embodiment, numerous implementation-specific decisions will be made to achieve the developer's specific goals, such as compliance with assembly-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

The system of the present application overcomes the abovementioned problems commonly associated with conventional ET tubes. Specifically, the preferred embodiment includes a connector in communication with both the primary and secondary passages. This feature eliminates the need for a lumen extending from the primary passage, as depicted in the McConnell device. The connector enables channeling of air, gas, anesthesia, and/or other associated gas/fluids to the patient during operation. The ET tube system also greatly reduces manufacturing cost due to the simplicity of design. Further detailed description and illustration of the different embodiments and methods are provided below and shown in the corresponding figures.

The embodiments contemplated in the present application can be utilized in different medical devices associated with channeling gases, fluids, drugs, and other substances to one or more locations within the human body. It will be appreciated that the contemplated embodiments are operably associated with both an ET tube system and an oral airway system. In one embodiment, the present device is an oral airway for insertion into the mouth and pharynx of a patient. The oral airway is adapted to connect to an anesthesia breathing connector, a suction tube, or a nasal cannula, interchangeably as needed, without necessitating the removal of the oral airway from the patient. In another embodiment, the device is an ET tube system configured to administer local drugs to one or more locations within the windpipe. It should be understood that the systems and devices discussed herein could easily be adapted for use with other related medical devices, and should not be narrowly construed solely for the embodiments discussed herein.

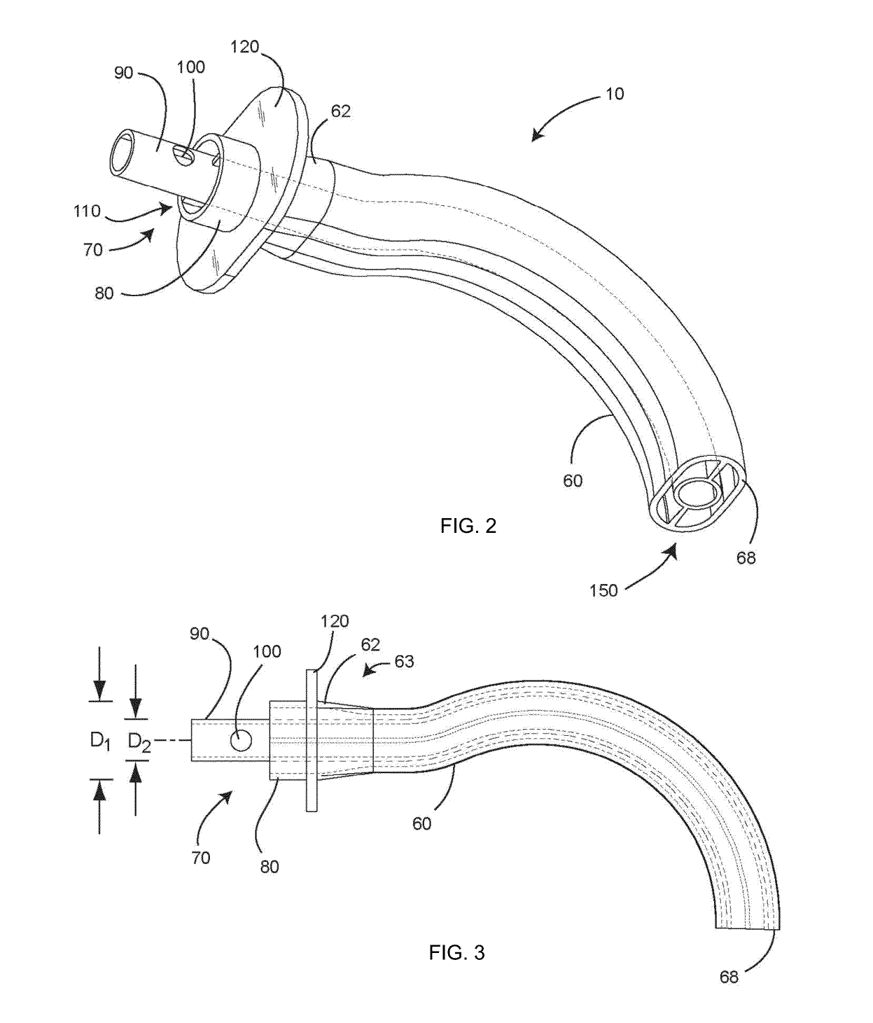

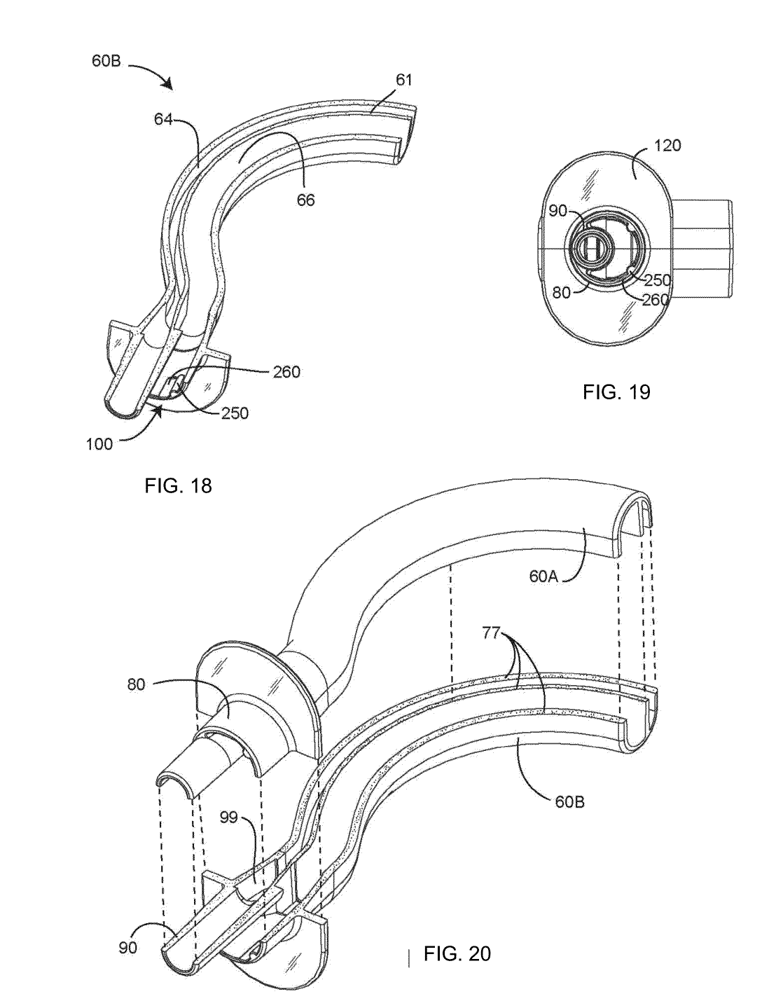

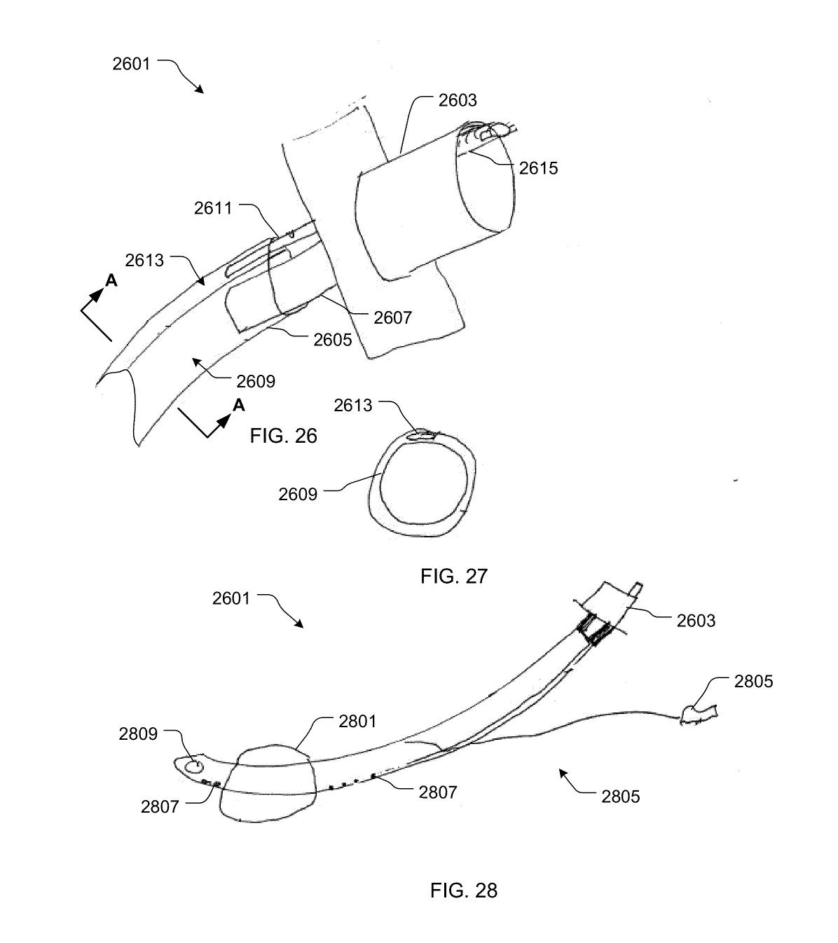

Referring now to the drawings, FIGS. 2-25 depict various views of the first embodiment, namely an oral airway system, while FIGS. 26-33 depict various views of a second contemplated embodiment, namely, an ET tube system. Both embodiments can be configured to utilize the connector discussed above.

The oral airway includes an arcuate hollow tubular member having a proximal end and a distal end adapted for inserting into the mouth and pharynx of the patient. A connector is fixed with the proximal end of the tubular member and includes a first portion that is adapted for fixing with the anesthesia breathing connector. A second portion of the connector is adapted for receiving the suction tube. At least one aperture is included in the second portion that is adapted for receiving a portion of the nasal cannula.

When the anesthesia breathing connector is connected to the first portion of the connector, the at least one aperture is sealed from the ambient atmosphere. Further, the anesthesia breathing connector is in fluid communication with the tubular member via at least one open gap formed between the first and second portions of the connector. A mouth guard extends outwardly from the connector.

In one embodiment of the invention, the tubular member includes a suction tube guide therein spanning substantially the length of the tubular member between the distal end thereof and the connector. The suction tube guide may be slidably positionable within the tubular member between a retracted position wherein the suction tube guide is substantially contained within the tubular member and an extended position wherein at least a portion of the suction tube guide extends past the distal end of the tubular member.

The present device is an oral airway that connects to an anesthesia breathing connector, a suction tube, or a nasal cannula, without necessitating the removal of the device from the patient. Further, the present invention, in one embodiment, allows for pinpoint depth adjustment of the suction tube. Using institutions will enjoy cost savings due to feature overlap with Yankauer suction tips, mask straps, and Laryngeal Mask Airways (LMAs), allowing in a percentage of procedures, discontinuance of use of some of these devices. Other features and advantages of the present invention will become apparent from the following more detailed description, taken in conjunction with the accompanying drawings, which illustrate, by way of example, the principles of the invention.

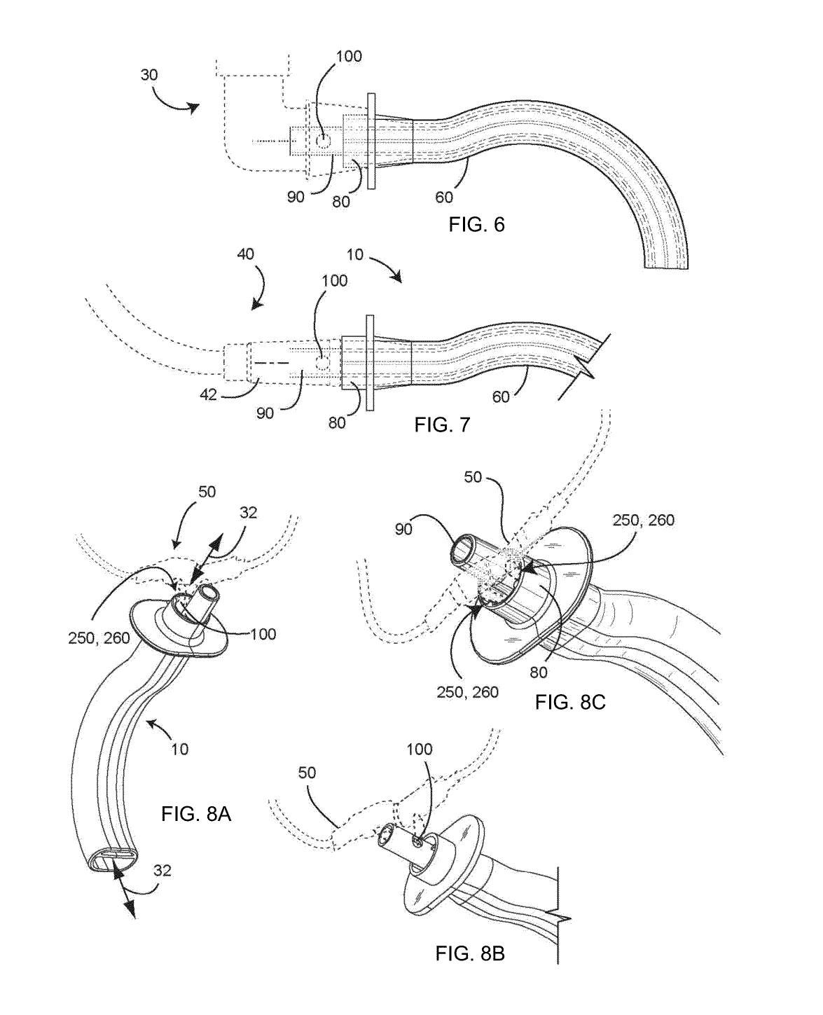

FIGS. 2 and 3 illustrate an oral airway 10 for insertion into the mouth and pharynx of a patient (not shown), thereby providing a breathing pathway 32 (FIG. 8A). The oral airway 10 is adapted to connect to either an anesthesia breathing connector 30 (FIG. 6), a suction tube 40 (FIG. 7), or a nasal cannula 50 (FIGS. 8A-8C), interchangeably as needed, without necessitating the removal of the oral airway 10 from the patient.

In FIG. 2, an arcuate hollow tubular member 60 has a proximal end 62 and a distal end 68. The tubular member 60 is adapted for inserting the distal end 68 first into the mouth and pharynx of the patient. Preferably the tubular member 60 is made from a non-toxic, semi-rigid plastic material.

In FIGS. 2-4, a connector 70 is fixed with the proximal end 62 of the tubular member 60 and includes a first portion 80 having a first diameter D1 (FIG. 3). The first portion 80 secures to the anesthesia breathing connector 30 (FIG. 6). A second portion 90 of the connector 70 has a second diameter D2 (FIG. 3) that is smaller than the first diameter Dl. The second portion 90 is adapted for receiving the suction tube 40 (FIG. 7) which may include a suction connector 42. Further, the anesthesia breathing connector 30 is in fluid communication with the tubular member 60 via at least one open gap 110 (FIGS. 2 and 5) formed between the first and second portions 80,90 of the connector 70. Preferably the connector 70 is made from a rigid, non-toxic plastic material. It should be noted that diameters D1 and D2 may be the proximal end diameters of connector 70 first and second portions 80,90 wherein those connector portions 80,90 are conical frustums such that anesthesia breathing connector 30 and suction connector 42 respectively adequately seal when affixed thereto.

The first and second portions 80,90 of the connector 70 are adapted to receive and retain therebetween a portion of the nasal cannula 50 (FIGS. 8A, 8B. and 11-18). In at least one embodiment, at least one protuberance 250 (FIGS. 8A, 8C, 16-22) between the first and second portions 80,90 is included and adapted for receiving and retaining therebetween a portion of the nasal cannula 50. Further, a groove 260 (FIGS. 8A, 8C, 16-22) may be included adjacent to each protuberance 250 for further facilitating the retention of the nasal cannula 50 between the first and second portions 80,90 of the connector 70.

In an alternate embodiment, at least one aperture 100 (FIGS. 2-7, 8B, 9 and 10) is included in the second portion 90 that is adapted for receiving a portion of the nasal cannula 50 (FIG. 8B). In such an embodiment, when the anesthesia breathing connector 30 is connected to the first portion 80 of the connector 70, the at least one aperture 100 is sealed from the ambient atmosphere, as illustrated in FIG. 6.

In FIGS. 2 and 3, a mouth guard 120 extends outwardly from the connector 70. The mouth guard 120 is preferably integrally formed with the connector 70, such as in a plastic injection molding process. Alternately, the mouth guard 120 may include a central aperture therein (not shown), whereby the mouth guard 120 being selectively fixable against the connector 70 by inserting the distal end 68 of the tubular member 60 to the central aperture of the mouth guard 120 and sliding the mouth guard 120 up the tubular member 60 to the connector 70. In yet another alternate embodiment, the mouth guard 120 includes a U-shaped slot (not shown) adapted for selectively removable attachment around an outer surface of the connector 70. The mouth guard 120 may further include mask strap prongs (not shown) at side edges thereof whereby an elastic strap (not shown) may be fixed to each mask strap prong and secured around the patient's head to retain the oral airway 10 in place on the patient. Preferably the mouth guard 120 is made from a rigid, non-toxic plastic material. The oral airway 10 may further include an integral bite guard region 63 on the distal side of the mouth guard 120 at the proximal end 62 of the tubular member 60 wherein the transition from the mouth guard 120 to the tubular member 60 includes increased wall thickness (FIG. 3).

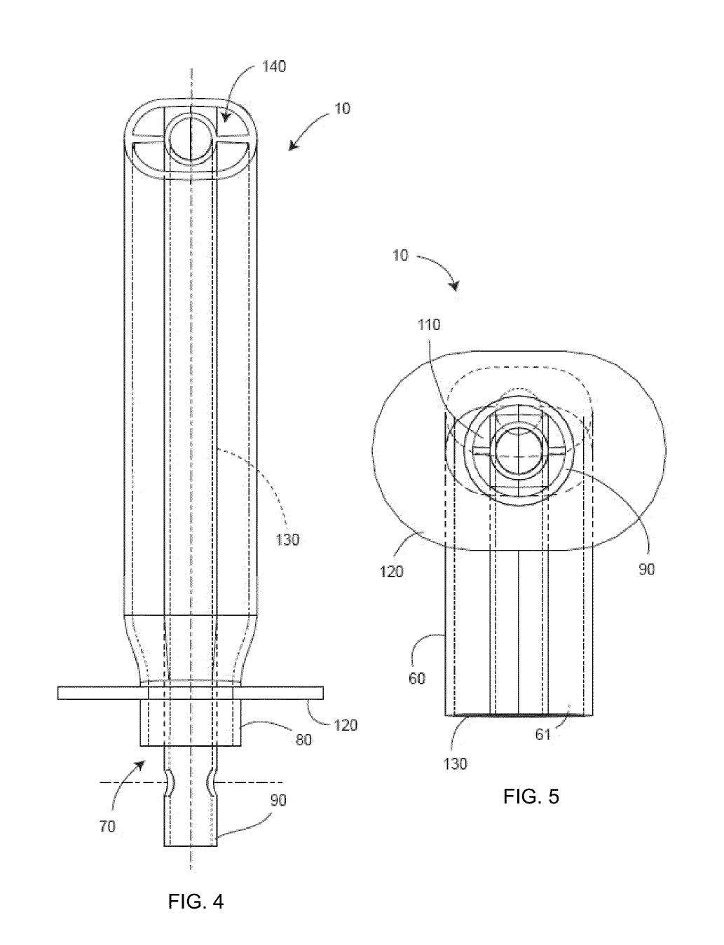

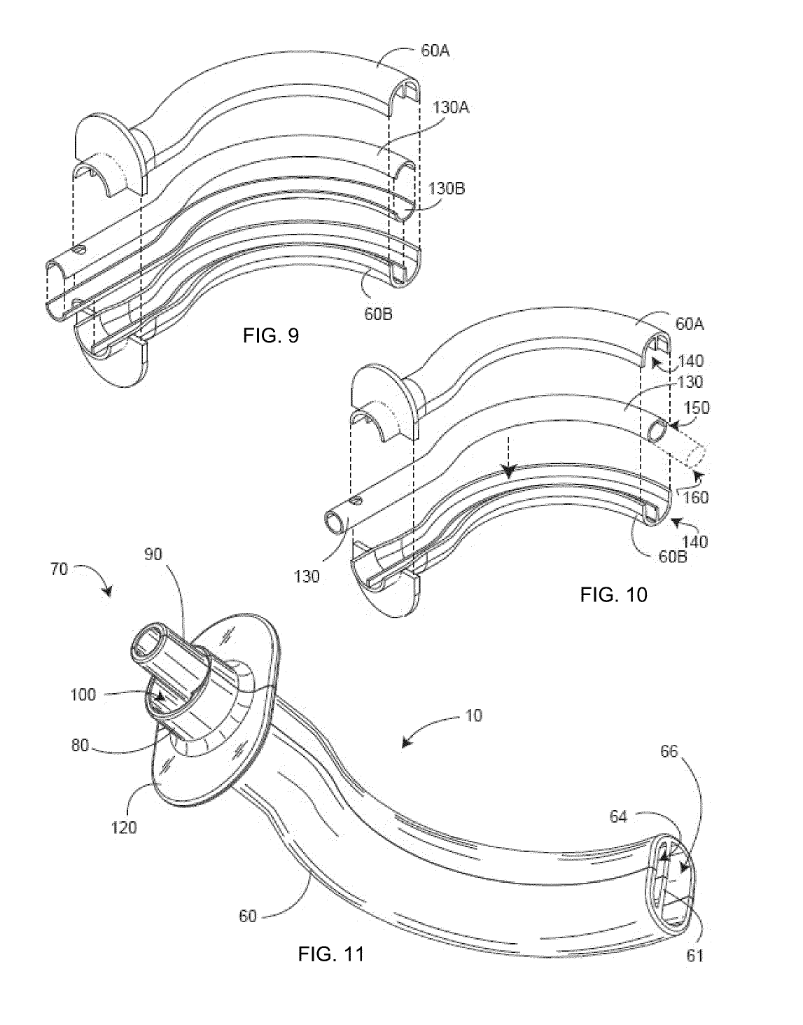

In one embodiment of the invention, the tubular member 60 further includes a suction tube guide 130 therein spanning substantially the length of the tubular member 60 between the distal end 68 thereof and the connector 70 (FIGS. 9 and 10). The suction tube guide 130 may be a length of flexible transparent tubing, for example (FIG. 10). A gap 140 between the suction tube guide 130 and the tubular member 60 allows airflow therebetween (FIGS. 4 and 10).

In one embodiment in FIG. 10, the suction tube guide 130 is slidably positionable within the tubular member 60 between a retracted position 150 wherein the suction tube guide 130 is substantially contained within the tubular member 60 and an extended position 160 wherein at least a portion of the suction tube guide 130 extends past the distal end 68 of the tubular member 60. In such an embodiment, the suction tube guide 130 may include at least one locking tab (not shown) at a proximal end thereof, and an internal wall of the tubular member 60 may include at least one locking tab groove (not shown) for capturing the tab between a locked position with the suction tube guide 130 in the retracted position 150, and an unlocked position wherein the suction tube guide 130 may be moved away from the retracted position 150 towards the extended position 160.

As illustrated in FIG. 9, the suction tube guide 130 may be formed as two halves 130A, 130B that are ultrasonically welded, solvent bonded, or otherwise mutually fixed together. The tubular member 60 may also be formed as two halves 60A,60B and ultrasonically welded, solvent bonded, or otherwise mutually fixed together around the suction tube guide 130 (FIG. 9). The suction tube guide 130 may alternately be a length of tubing that is captured between two halves 60A,60B of the tubular member 60 (FIG. 10) which is then ultrasonically welded or otherwise fixed therearound.

Alternately, in an alternate embodiment (not shown), the suction tube guide 130 and the first portion 80 of the connector 70 are mutually fixed together and both slidably positionable together within the tubular member 60 between the retracted position, wherein the suction tube guide 130 is substantially contained within the tubular member 60, and the extended position, wherein at least a portion of the suction tube guide 130 extends past the distal end 68 of the tubular member 60. In such an embodiment, the first portion 80 of the connector 70 may include at least one of the locking tabs, and wherein the second portion 90 of the connector 70 may include at least one of the locking tab grooves for capturing the locking tab between the locked position and the unlocked position. As such, the first portion 80 of the connector 70 and the suction tube guide 130 may be moved together away from the retracted position towards the extend position.

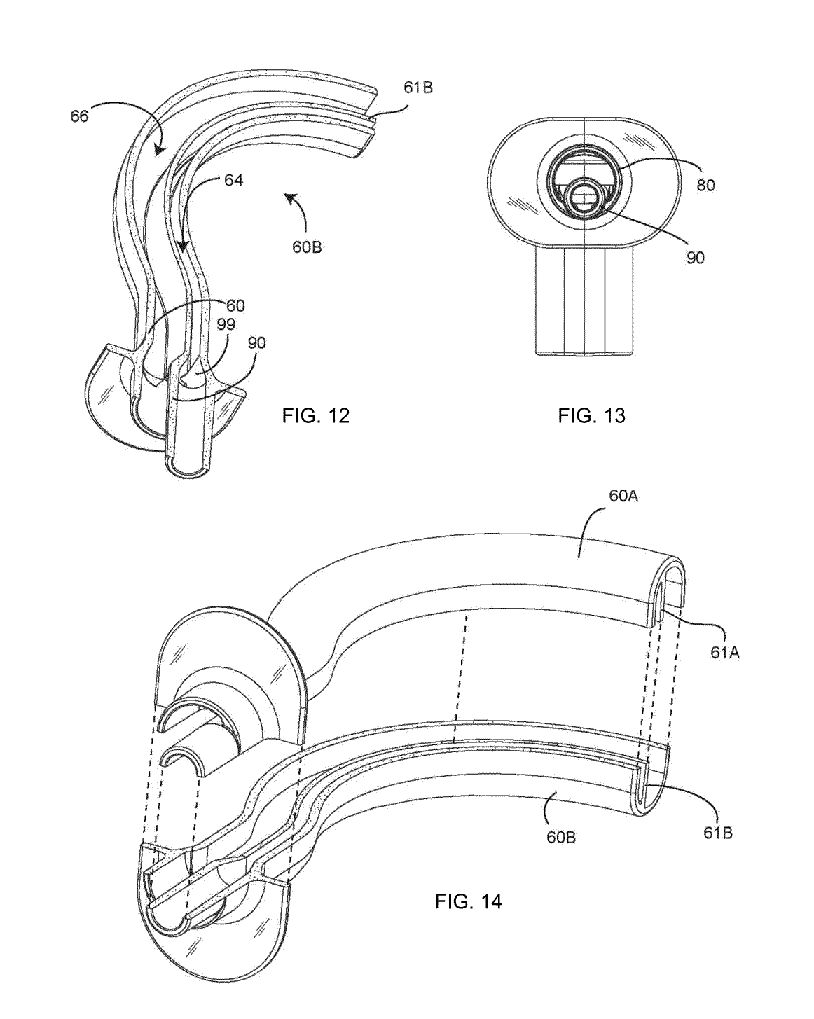

In one alternate preferred embodiment (FIGS. 11-14), the oral airway 10 is formed as two halves that divides the tubular member 60 into two halves 60A,60B that are fixed together with ultrasonic welding, solvent bonded, or otherwise mutually fixed together.

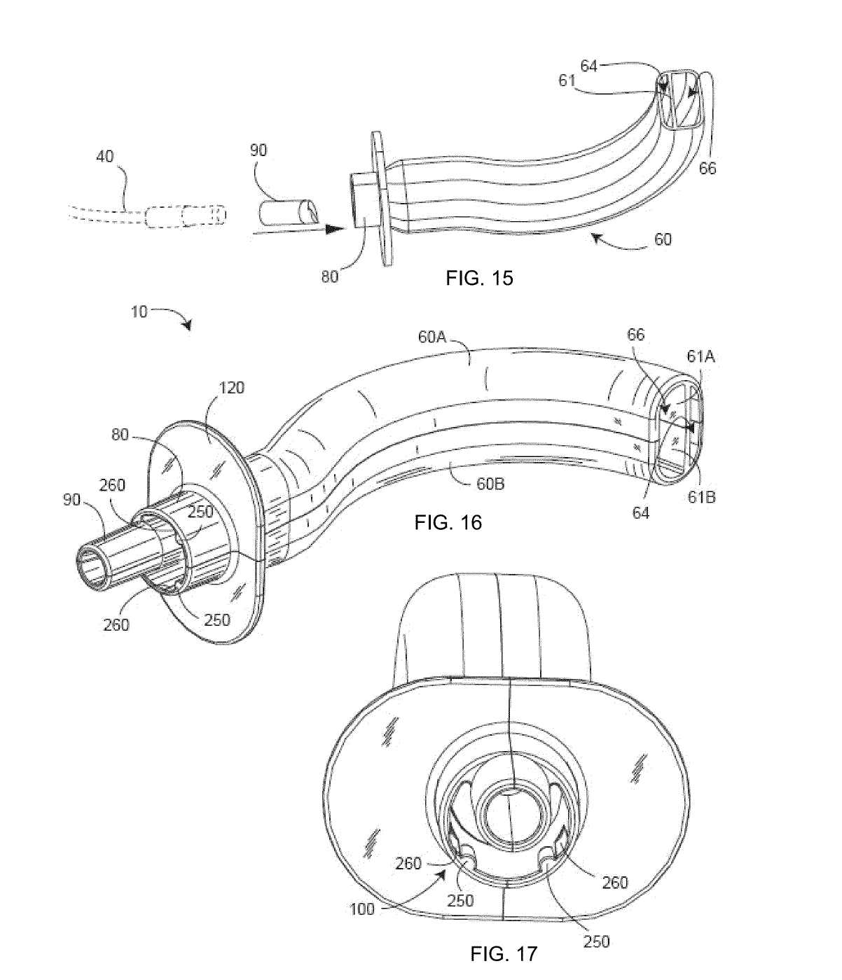

Each half 60A,60B of the tubular member 60 further including one half 6ZA,61B of a conduit separator 61 (FIGS. 11-12 and 14) that spans the length of the tubular member 60 and bifurcates the tubular member 60 into a suction conduit 64 and a breathing conduit 66. In FIG. 12, the second portion 90 of the connector 70 may include a funnel 99 that is in fluid communication with only the suction conduit 64. As such, in FIG. 15, when the suction tube 40 is fixed within the second portion 90 of the connector 70, only the suction conduit 64 is in fluid communication therewith, and not the breathing conduit 66. Such an embodiment may be formed by fixing together each half 60A,60B of the tubular member 60, and then fixing the second portion 90 of the connector 70 into the first portion 80 of the connector 70, such that the funnel 99 is aligned with the suction conduit 64, with a suitable cement adhesive or ultrasonic welding method, as illustrated in FIG. 15. In an alternate preferred embodiment, the location of the suction conduit 64 and the breathing conduit 66 may be reversed, as illustrated in FIGS. 16-20, with the location of the second portion 90 of the connector 70 reversed accordingly. In one situation, when the patient is lying face up, it is preferable to locate the suction conduit towards a back side of a patient's throat where fluid may be gathering. In another situation, when the patient is lying face down, it is preferable to locate the suction conduit towards a front side of a patient's throat where fluid may be gathering.

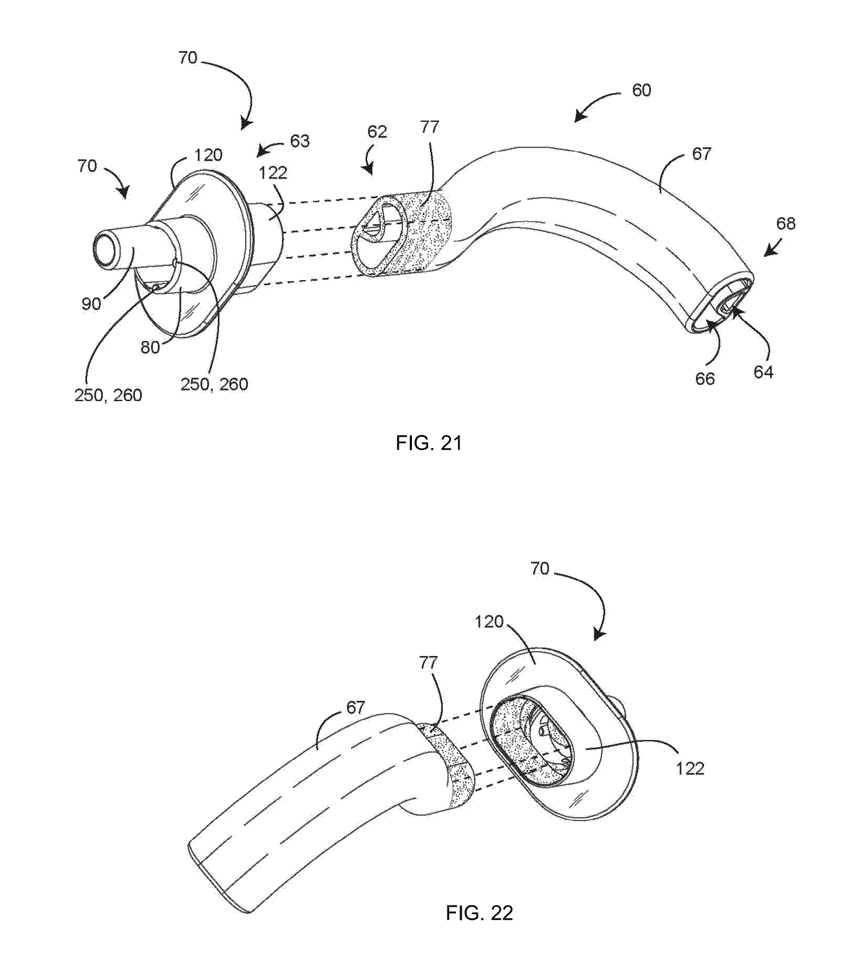

FIGS. 21 and 22 illustrate yet another preferred embodiment wherein the tubular member 60 is an arcuate multi-lumen extruded tube 67 and connector 70 includes a tube socket 122 adapted for receiving the proximal end 62 of the multi-lumen extruded tube 67. In one embodiment, the connector 70 and tubular member 60 are fixed together with a solvent bonding process, in particular using a solvent bond 77. In another embodiment, the connector 70 and the multi-lumen extruded tube 67 are fixed together with a permanent adhesive, or ultrasonic welding, or the like. The tube socket 122 may further serve as a bite guard 63.

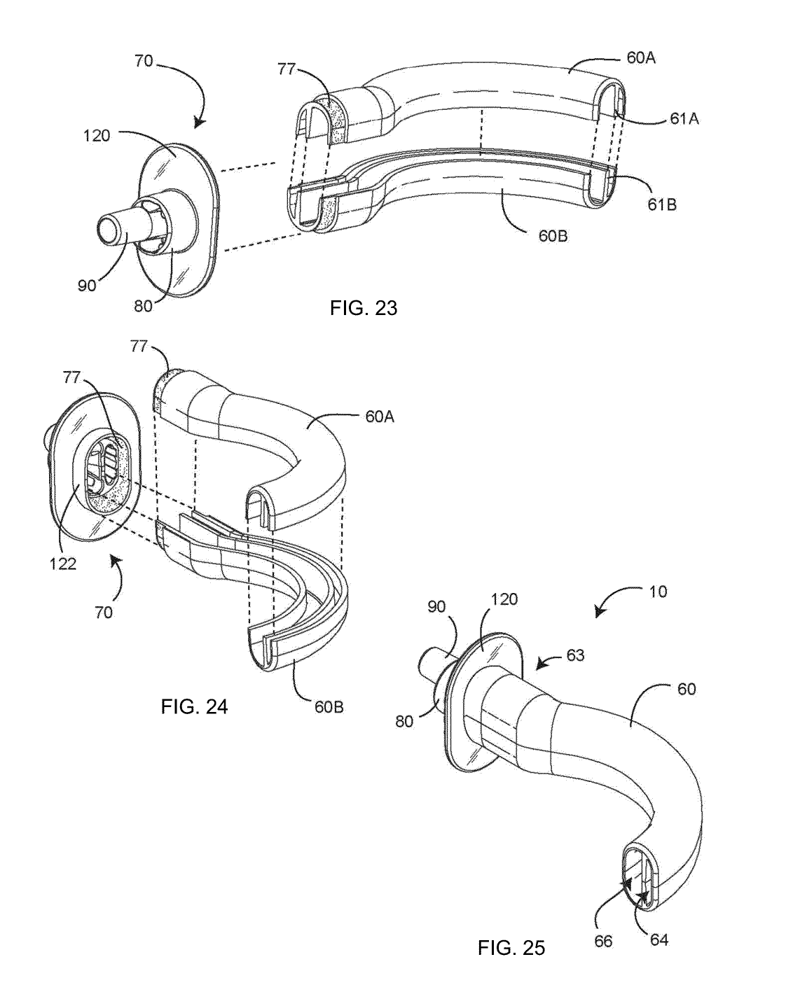

Alternatively, an oral airway (FIGS. 23-25) comprises three injection-molded parts, namely the connector 70 with the socket 122, and tubular member 60 formed as two halves 60A,60B. One advantage of such an embodiment is that the injection molds (not shown) for such parts may be relatively simple, without any side pulls or the like.

While a particular form of the invention has been illustrated and described, it will be apparent that various modifications can be made without departing from the spirit and scope of the invention. For example, three types of connectors are illustrated as cooperating with the oral airway of the present invention, but other types of conduit connectors may also be adapted for use therewith, for example, the connectors discussed below. Accordingly, it is not intended that the invention be limited, except as by the appended claims.

Referring now to FIGS. 26-33 in the drawings, various views of an ET tube system 2601 are shown according to one preferred embodiment of the present application. ET tube 2601 provides effective means for administering local anesthesia and/or other desired gas/liquid to one or more locations within the trachea. It is contemplated utilizing one or more of the features of system 2601 in the above embodiments discussed herein, and likewise, the features discussed above can be included in system 2601.

Referring specifically to FIG. 26 in the drawings, a partial oblique view of system 2601 is shown. System 2601 comprises one or more of a connector 2603 secured to an ET tube 2605. Connector 2603 is configured to secure with a breathing apparatus and/or other operably associated devices (not shown). Connector 2603 preferably includes a first conduit 2607 that fits within and in gas/fluid communication with a primary passage 2609 of ET tube 2605. Connector 2603 further includes a second conduit 2611 that fits within and in gas/fluid communication with a secondary passage 2613 of ET tube 2605. In the preferred embodiment, first conduit 2607 remains fluidly separated from second conduit 2611.

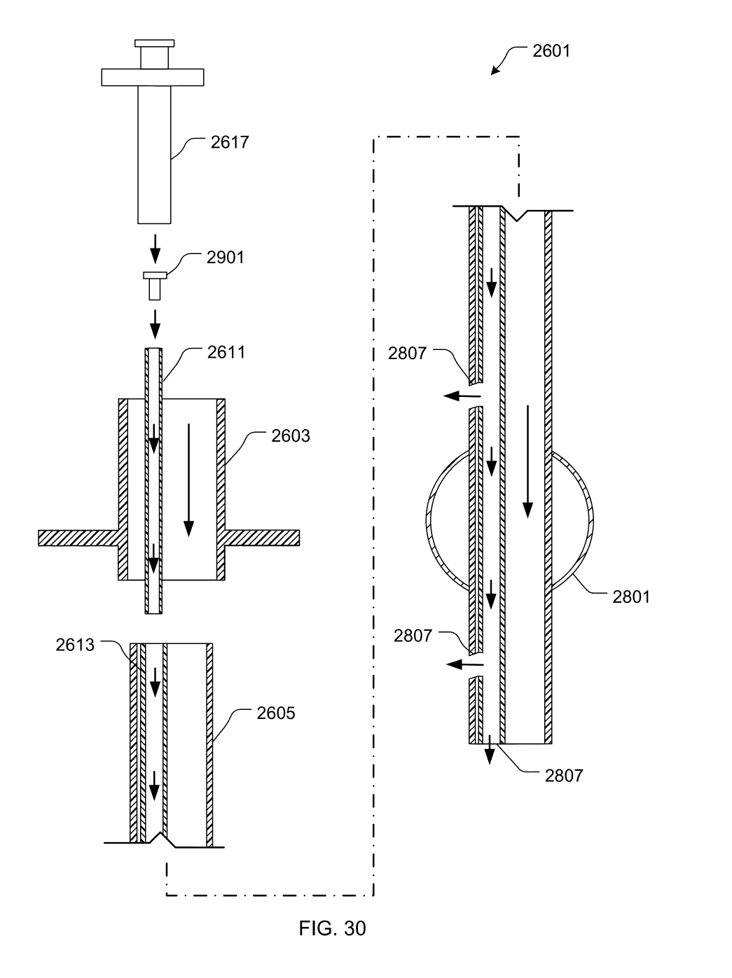

Connector 2603 further comprises an attachment device 2615 in fluid communication with second conduit 2611. Attachment device 2615 provides effective means to secure with a syringe 2617 and/or other operably associated device for administering fluids within conduit 2611 (see FIG. 30). Attachment device 2615 is preferably a luer lock that threadingly engages with the syringe. Device 2615 enables easy and rapid attachment of the syringe during administration of the anesthesia. However, it will be appreciated that alternative embodiments could include other devices in lieu of a luer lock, for example, an attachment device 2619 (see FIG. 33) configured to receive a syringe needle (not shown). Additional embodiments can also include a one-way solenoid valve 2621 in gas/fluid communication with second conduit 2611 as means to prevent gas/fluid backflow. Although shown exposed from the inner cavity of connector 2603, attachment device 2615 can be completely disposed therein or flush with the opening of connector 2603.

During use, ET tube 2605 is positioned within the trachea of the patient and secured therein with a balloon 2801 according to one or more methods known to a person skilled in the art. Thereafter, connector 2603 is secured to ET tube 2605, such that first conduit 2607 and second conduit 2611 fit snugly within respective passages 2609 and 2613. In the contemplated embodiment, the respirator adapter is removably attached to the outer periphery of connector 2603. During operation, the respirator adapter is removed when access to second conduit 2611 is required, for example, administering local anesthesia. However, alternative embodiments could include access to the second conduit without removing the respirator adapter (see FIG. 32).

In one embodiment, system 2601 describes a novel, useful, and non-obvious way of delivering local anesthesia along the length of the ET tube without the need for an extraneous separate cannulas, redundant ports, cumbersome y-adapters, more than one balloon or the need to remove the breathing circuit connector in order to access a lumen for medicine administration. It will be appreciated that the manufacturing of the ET tube system with the ability to provide laryno-tracheal anesthesia will be more cost effective with the features discussed herein.

It will be appreciated that various embodiments are discussed herein, including the features of placing an attachment means located outside rather than within the connector in addition to an attachment means not being present at all, and instead a standard IV catheter hub attached to a syringe can be friction fit into the open aperture that communicates with the lumen for administration of local anesthesia.

In the preferred embodiment, the manufacturing of system 2601 includes the process of injection molding the connector as one piece from elastomeric, rigid material such as polyvinyl chloride (PVC). The breathing circuit has two lumens—one for oxygen/medical gas and one for local anesthesia administration. The luer lock is molded in a separate step. Alternatively, the luer lock may also be molded in one step as part of the connector, but may require molding side pull action. The ET tube is preferably co-extruded or extruded as a mulit-lumen tube form flexible PVC or silicone. Post manufacturing assembly requires the luer lock to be fitted into the connector with can be solvent bonded, ultrasonically welded, friction fitted, or snap-fitted by molded retention hooks or any combination thereof.

In FIG. 27, a cross-sectional view of ET tube 2605 is shown taken at A-A of FIG. 26. In this contemplated embodiment, secondary passage 2613 is disposed within the wall of primary passage 2609. However, it will be appreciated that secondary passage 2613 can be positioned within the interior of passage 2609 in lieu of the exemplary embodiment.

FIG. 28 shows an oblique view of system 2601 as assembled. System 2601 is optionally provided with a tube 2803 in gas communication with balloon 2801. A connector securely fastens to tube 2803 and is configured to provide channeling means for inflating balloon 2801.

System 2601 further comprises a plurality of ports 2807 positioned upstream and/or downstream of balloon 2801. Ports 2807 are in fluid communication with secondary passage 2613 and are configured to provide administration of anesthesia and/or other substances at selected locations. The size, number, and location of ports 2807 are determined according to desired applications. It will also be appreciated that the Murphy's eye 2809 could also be in fluid communication with secondary passage 2613 for administering drugs.

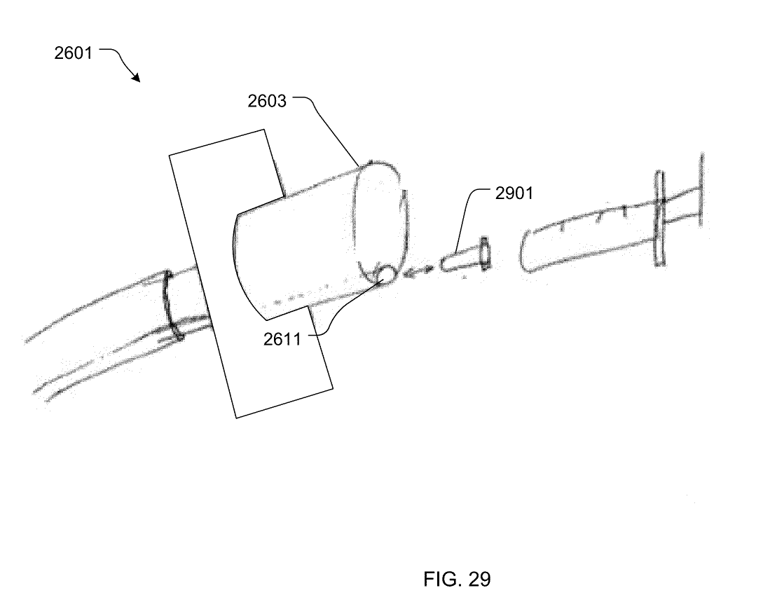

In FIG. 29, an oblique view of an alternative embodiment of system 2601 is shown. In this embodiment, an attachment device 2901 is friction fitted in second conduit 2611. It will be appreciated that the attachment device can either be permanently secured to second conduit 2611 or snugly fit therein.

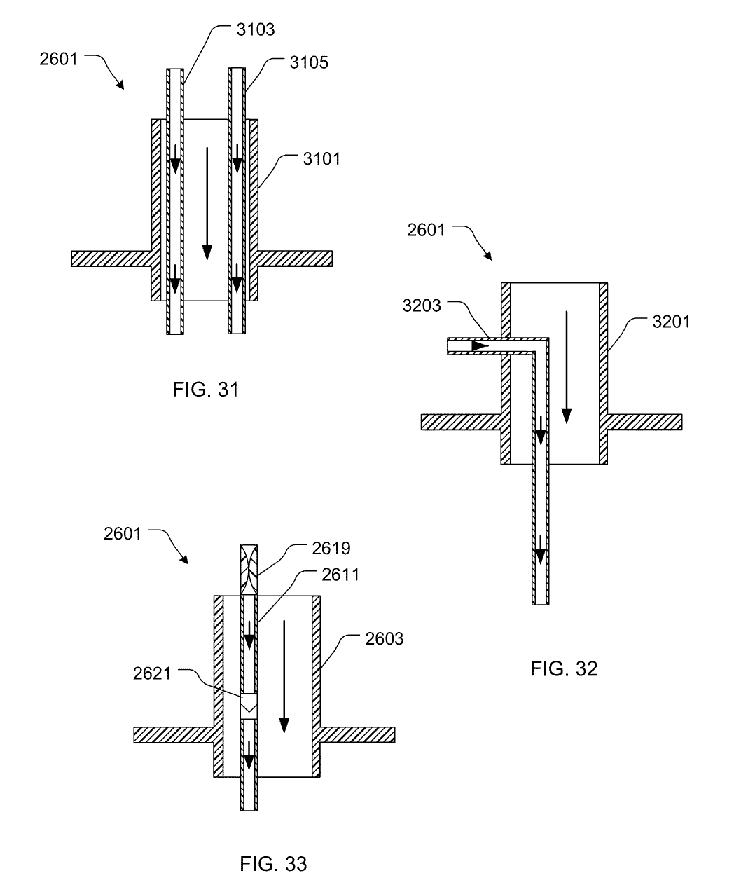

FIG. 30 depicts a cross-sectional view of system 2601 for viewing access of the primary and secondary passages. For clarity, arrows are utilized to depict movement of the gas/fluid being channeled through the various conduits and passages. It will be appreciated that a third conduit and associated passage can be utilized in conjunction with one or more ports for administering local gas/fluid at different locations. Further, the third passage can also be in gaseous communication with the balloon cuff. This feature eliminates the need for a lumen to extend from the primary passage during inflation (see FIG. 31).

Turning next to FIGS. 31-33, alternative embodiments of connector 2603 are shown. It is contemplated interchanging the features of the different embodiments.

In FIG. 31, connector 3101 includes two conduits 3103 and 3103 configured to fit within two passages disposed within the primary passage of the ET tube (not shown). This embodiment enables multiple administering of gas/fluids to different separated ports selectively located along the ET tube. Further, this embodiment also allows inflating the balloon without the need for a tube extending from the ET tube itself.

FIG. 32 depicts an alternative embodiment, wherein a connector 3201 utilizes a second conduit 3203 that extend from an inner wall of connector 3201. This embodiment allows the user to administer gas/fluid to the second conduit without removing the respirator adapter from connector 3201.

FIG. 33 depicts an alternative embodiment of connector 2603 having the attachment means and solenoid discussed above.

It will be appreciated that alternative embodiments are contemplated wherein there is no plug or a hub as discussed above. Further, the small aperture for local anesthesia administration can mate directly with the distal tip of any standard syringe—with or without Luer lock. Friction fit by the user keeps the two mated together during local anesthesia administration. In another embodiment, the small aperture remains open without solenoid or plug. It should also be appreciated that no airleak will occur if distal apertures above the ETT balloon are kept to a minimum (i.e. 1 or 2 holes) because positive pressure ventilation will preferentially flow via the path of least resistance—the main ETT oxygen/gas lumen.

It will be appreciated that the features discussed above are applicable for use with a Laryngeal Mask Airway and/or a supraglottic airway device, and the connector can also be manufactured in one step (one entire/whole part instead of two halves) by 3D printing technology and the solenoid, one-way valve added later in another step (glue, adhesive, as previous, etc).

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms.

Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. An endotracheal tube system, comprising:
an endotracheal tube having a first conduit and a second conduit, the first conduit being fluidly separable from the second conduit; and
a connector in communication with both the first conduit and the second conduit;
wherein the endotracheal tube extends within a mouth and pharynx of a patient to provide a breathing pathway;
wherein the connector is configured to cooperate with either an anesthesia breathing apparatus, a suction tube, or a nasal cannula; and
wherein either the anesthesia breathing apparatus, the suction tube, or the nasal cannula is utilized on the patient without necessitating the removal of the oral airway from the mouth and pharynx.
